(19) United States
(12) Patent Application Publication
(10) Pub. No.: US 2014/0066854 A1
(43) Pub. Date: Mar. 6, 2014

(54) CATHETER TUNNELER WITH LARIAT MEMBER

(71) Applicants: Peter C. MARTIN, Assonet, MA (US); Steve B. COTE, Mendon, MA (US); Dillon BURGESS, Long Beach, NY (US)

(72) Inventors: Peter C. MARTIN, Assonet, MA (US); Steve B. COTE, Mendon, MA (US); Dillon BURGESS, Long Beach, NY (US)

(73) Assignee: TYCO HEALTHCARE GROUP LP, Mansfield, MA (US)

(21) Appl. No.: 13/605,388

(22) Filed: Sep. 6, 2012

(51) Int. Cl. A61B 17/34

(52) U.S. Cl. 604/164.01

(57) ABSTRACT

A tunneler device is provided which includes a handle, a shaft supported on distal end of the handle, and a body positioned on a distal end of the shaft. The body supports an extension segment and a lariat member. The lariat member includes a rod that supports a capturing element. The capturing element is at least partially positioned about a perimeter of the extension segment in a spaced apart relationship with the extension segment, wherein the extension segment and the capturing element define an annular channel dimensioned to receive a catheter.

### BACKGROUND

1. Technical Field

The present disclosure relates generally to indwelling catheters, and more particularly to a tunneler device for preparing a subcutaneous tunnel for placement of an indwelling catheter.

2. Background of Related Art

Catheters are flexible instruments which facilitate the withdrawal and introduction of fluids from and to body cavities, ducts, and vessels. Typically, a distal end of the catheter is implanted into, for example, the vasculature of a patient to withdraw blood from the patient or introduce medicaments into the patient. When a catheter is implanted into the vasculature of a patient, the catheter's distal portion is inserted through an incision in the patient's skin and through an opening in the vasculature (venotomy site) until the distal tip is precisely located at a desired site. The proximal portion of the catheter remains external of the patient to provide access to the catheter for infusion and/or withdrawal of fluids such as for hemodialysis. Often, the proximal portion of the catheter is directed away from the venotomy site to a remote site through a subcutaneous tunnel such that the catheter can be comfortably positioned on a patient's body for long term use. To accomplish this, a tunneler device is typically secured to the proximal portion of the catheter and the catheter is pulled by the tunneler device through the subcutaneous tunnel. The tunneler device usually also creates the subcutaneous tunnel.

Tunneler devices have one end which is configured to securely engage the proximal end of the catheter. Typically, the engagement end of the tunneler device includes at least one extension segment which is dimensioned to be received within a lumen or lumens of the catheter. Although this provides somewhat effective connection of the tunneler device to the catheter, detachments are known to occur during a subsequent tunneling procedure. Such detachments require additional steps and therefore increases the duration of the catheter placement procedure, as well as increase the risk associated with the procedure, such as excessive trauma to the patient.

It would be beneficial to have a tunneling device including a catheter securement device which is capable of improved securement of the tunneler device to the catheter to prevent detachment during a tunneling procedure.

### SUMMARY

Accordingly, a tunneler device is provided. In general, in one aspect of the present disclosure, a tunneler device includes a handle, a shaft extending distally from the handle, and a connecting assembly including a body positioned on a distal end of the shaft.

The body supports an extension segment and a lariat member. The extension segment is configured to be received in a lumen of a catheter for engagement with an inner surface of the catheter. The lariat member includes a rod and a capturing element. The rod of the lariat member and the extension segment may define parallel axes. The rod extends distally from the body and supports the capturing element. The capturing element is at least partially positioned about a circumference of an outer surface of the catheter and is configured to enable movement of the catheter to a position about the extension segment but to obstruct movement of the catheter from about the extension segment. The capturing element of the lariat member may encircle the extension segment. The capturing element may include an inwardly tapering distal surface that defines an opening configured and dimensioned to enable movement of the catheter about the extension segment. The opening may be substantially circular. The capturing element defines a proximal edge positioned to engage the catheter to obstruct movement of the catheter from the extension segment.

The tunneler device includes a cap that is selectively engageable with the connecting assembly. The body of the connecting assembly includes a threaded segment with an external thread and the cap includes an internal thread such that the internal thread of the cap and the external thread of the threaded segment of the body are selectively threadably engageable to secure the cap to the connecting assembly. The cap encloses the extension segment and the lariat member when the cap is secured to the connecting assembly.

According to another aspect of the present disclosure, a tunneler device includes a handle, a shaft supported on a distal end of the handle, and a body positioned on a distal end of the shaft.

The body supports an extension segment and a lariat member. The lariat member includes a rod that supports a capturing element. The capturing element is at least partially positioned about a perimeter of the extension segment in spaced apart relationship with the extension segment, wherein the extension segment and the capturing element define an annular channel dimensioned to receive a catheter. The rod of the lariat member and the extension segment may define parallel axes. The capturing element may include a distal inwardly tapering surface that defines an opening configured and dimensioned to receive the catheter. The opening may be substantially circular. The capturing element defines a proximal edge positioned to engage the catheter to obstruct movement of the catheter from the extension segment.

The tunneler device includes a cap that is selectively engageable with the body. The body includes a threaded segment with an external thread and the cap includes an internal thread such that the internal thread of the cap and the external thread of the threaded segment of the body are selectively threadably engageable to secure the cap to the body. The cap encloses the extension segment and the lariat member when the cap is secured to the body.

According to yet another aspect, a tunneler device includes a handle, a shaft extending distally from the handle, a connecting assembly including a body positioned on a distal end of the shaft, and a cap.

The body supports an extension segment and a lariat member. The extension segment is configured to be received in a lumen of a catheter for engagement with an inner surface of the catheter. The lariat member includes a rod and a capturing element. The rod extends distally from the body and supports the capturing element. The capturing element surrounds an outer surface of the catheter and is configured to enable movement of the catheter to a position about the extension segment but to obstruct movement of the catheter from about the extension segment. The capturing element includes a distal inwardly tapering surface that defines an opening configured and dimensioned to receive the catheter.

The cap is selectively attachable to the body of the connecting assembly to enclose the extension segment and the lariat member for facilitating passage of the tunneler device through a subcutaneous tunnel. The cap encloses the extension segment and the lariat member when the cap is secured to the body.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a side view of a tunneler device according to the present disclosure;

FIG. 2 is a side view of the presently disclosed tunneler device and a proximal region of a catheter, with a cap of the presently disclosed tunneler device being removed from the presently disclosed tunneler device;

FIG. 3 is a side view of the presently disclosed tunneler device and the catheter shown secured together;

FIG. 4 is an enlarged perspective view of a distal region of the presently disclosed tunneler device and a proximal region of the catheter, with the cap of the presently disclosed tunneler device being removed from the presently disclosed tunneler device;

FIG. 4A is an enlarged cross-sectional view of a lariat member of the presently disclosed tunneler and a proximal region of the catheter; and

FIGS. 5-8 are progressive views illustrating a tunneling procedure with the presently disclosed tunneler device.

### DETAILED DESCRIPTION

The embodiments of the present disclosure are directed to a tunneling system including a catheter and a tunneling device for property positioning the catheter within a patient. The tunneling system of the present disclosure may have various medical applications. For example, during a hemodialysis catheter implantation procedure, the tunneling device may be manipulated by a clinician to create or enlarge a subcutaneous tunnel within a patient to properly position the catheter on the patient. It is envisioned that the presently disclosed tunneling system may be employed in any other suitable procedure.

In the discussion that follows, the term “clinician” refers to a doctor, a nurse, or any other care provider and may include support personnel. The term “proximal” refers to the portion of a structure that is closer to a clinician, whereas the term “distal” refers to the portion of a structure that is farther from the clinician.

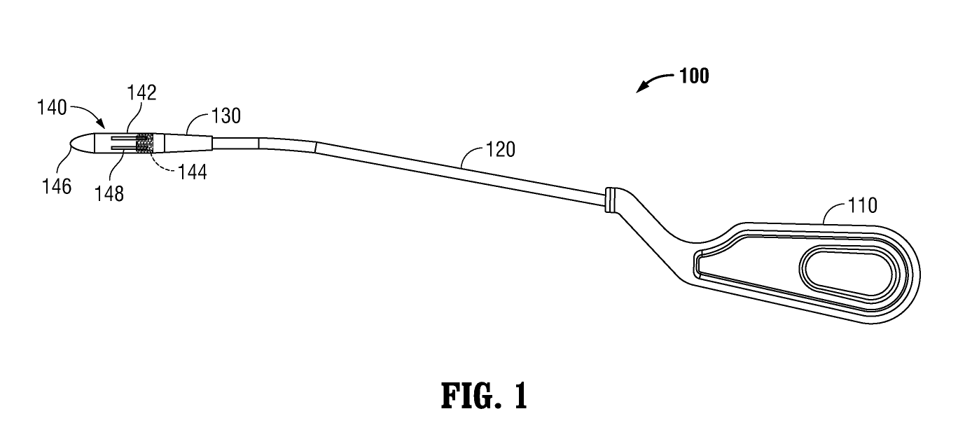

With reference to FIG. 1, a tunneler device according to the present disclosure is shown generally as tunneler device 100. Although tunneler device 100 will be described for use in the placement of a dialysis catheter within a patient, tunneler device 100 may be used for the placement of various other medical implements. Tunneler device 100 includes a handle 110, a shaft 120 extending from handle 110, a connecting assembly 130 mounted on a distal end of shaft 120, and a tunneling cap 140 selectively engageable with connecting assembly 130. Any or all of the components of tunneler device 100 may be sterilizable, and thus, reusable. Alternatively, tunneler device 100 may be disposable.

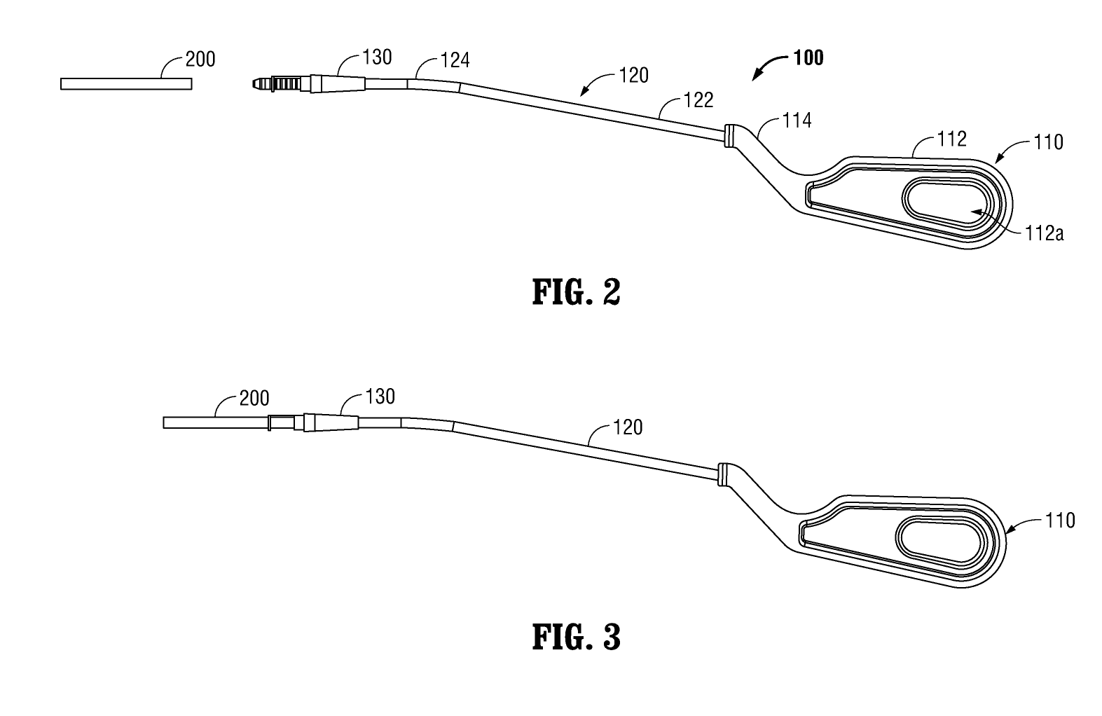

Turning now to FIG. 2, handle 110 is supported on a proximal end of tunneler device 100 and is configured for operable engagement by a clinician. In particular, handle 110 includes a loop 112 defining an opening 112a at a proximal end of handle 110 dimensioned to receive a clinician's fingers. A distal handle segment 114 is provided at a distal end of handle 110 that supports a proximal end of shaft 120. Distal handle segment 114 is obliquely arranged with respect to shaft 120 at any suitable angle to displace or offset the hand of the clinician relative to the patient during use in a tunneling procedure. Handle 110 may include ridges, bumps, knurls, and/or any other suitable non-slip features configured to facilitate gripping engagement by a clinician. Handle 110 may also be configured for operable engagement with a robotic arm (not shown) or other mechanical manipulating device.

A proximal end of shaft 120 is secured to, and may be embedded within, distal handle segment 114 of handle 110 to connect handle 110 and shaft 120 together. Shaft 120 extends distally from a distal end of handle 110 and includes a proximal shaft segment 122 and a distal shaft segment 124. In one embodiment, handle 110 is molded about a proximal end of proximal shaft segment 122 of shaft 120. Alternatively, shaft 120, handle 110, and connecting assembly 130 may be unitarily formed. Distal shaft segment 124 may be obliquely arranged with respect to proximal shaft segment 122 at any suitable angle to facilitate formation of a tunnel in a patient as desired. Further, shaft 120 may be malleable to enable the clinician to bend shaft 120 as needed for a particular procedure. For example, the clinician may bend shaft 120 into a curved shape to enable tunneler device 100 to create a curved or arced tunnel.

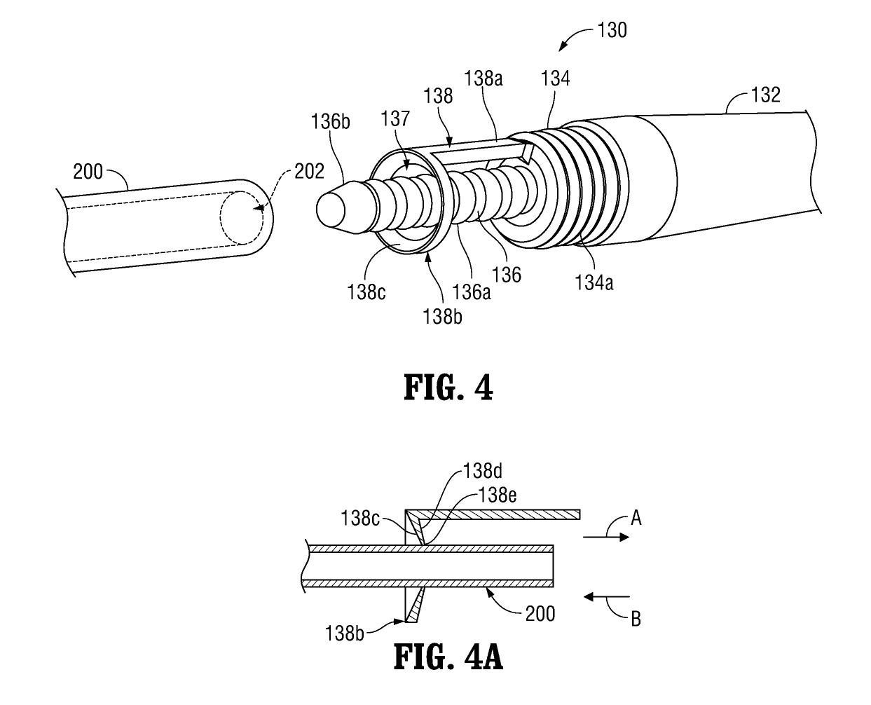

Turning now to FIG. 4, connecting assembly 130 is supported on a distal end of shaft 120 and includes main body 132 for supporting connecting assembly 130 on the distal end of shaft 120, a threaded segment 134 for releasably engaging tunneling cap 140, an extension segment 136 for engaging a catheter 200 (with one or more lumens), and a lariat member 138 for supporting catheter 200 on extension segment 136. As appreciated, connecting assembly 130 may be integrally formed with shaft 120, or, in the alternative, secured to shaft 120 using known fastening techniques such as overmolding.

With continuing reference to FIG. 4, threaded segment 134 includes one or more external threads 134a positioned adjacent to the main body 132 that facilitate a threaded engagement with one or more internal threads 144 (see FIG. 1) of tunneling cap 140. Alternatively, other connections may be made between the tunneling cap 140 and the connector assembly 130 to secure the tunneling cap 140 thereto. Extension segment 136 extends distally from the main body 132. In the illustrated embodiment, the extension segment 136 extends from the threaded segment 134 and includes a plurality of barbs 136a and a blunt tip 136b. Although illustrated as spherically shaped barbs 136b, other barb configurations are envisioned. Extension segment 136 is dimensioned to be received within a lumen 202 of catheter 200 when tunneling cap 140 is removed from connecting assembly 130 such that barbs 136a frictionally engage an inner surface of the catheter 200 to secure extension segment 136 to the catheter 200.

Lariat member 138 may include at least one elongate rod 138a and a capturing element 138b supported on a distal end of the elongate rod 138a or at any suitable location along elongate rod 138a. A proximal end of the elongate rod 138a may be mounted to a distal end of threaded segment 134 so that elongate rod 138a extends distally from threaded segment 134. Capturing element 138b of lariat member 138 is positioned about the circumference of extension segment 136 in a spaced apart relationship with extension segment 136. Capturing element 138b may extend only partially about the perimeter of extension segment 136, for example, the path may extend 90 degrees, 180 degrees, or more about the circumference of extension segment 136. While an elongated rod 138a is shown, the capturing element 138b may be connected to the main body 132 in any suitable fashion. For example, the capturing element 138b may be supported by multiple rods, a cylinder or a partial cylinder extending from the main body 132.

Referring also to FIG. 4A, capturing element 138b includes an inwardly tapering distal surface 138c and an angled proximal surface 138d defining a proximal edge 138e. Tapering distal surface 138c and angled proximal surface 138d may be disposed at any suitable angle to dispose proximal edge 138e at any suitable angle. Capturing element 138b defines an opening 137 dimensioned to receive the catheter 200 therethrough when extension segment 136 is inserted within a lumen 202 of catheter 200 (see FIG. 3). As such, catheter 200 will be received about extension segment 136 within opening 137 of lariat member 138. Opening 137 may be any suitable shape including, for example, circular, oval, or polygonal. With reference again to FIG. 3, capturing element 138b is positioned about the outer circumference of the catheter 200 when catheter 200 is secured between extension segment 136 and lariat member 138. As illustrated in FIG. 4A, tapering distal surface 138c of capturing element 138b enables catheter 200 to be slid in the direction of arrow “A” into opening 137, but proximal edge 138e obstructs catheter 200 from being slid from lariat member 138 in the direction indicated by arrow “B.” Any attempt to separate catheter 200 from lariat member 138 will cause proximal edge 138e of lariat member 138 to dig into an outer surface of catheter 200 to obstruct the separation of catheter 200 from lariat member 138. The lariat member 138 may be made of any suitable material. In one embodiment, the lariat member 138 is made of a resiliently deformable material, which, when combined with the shape of capture element 138, will further enhance the retention of the catheter 200. Specifically, the resilient material and shape will enable the proximal edge 138e to move proximally as the catheter 200 is inserted, thereby providing easier insertion. However, as the catheter 200 is pulled distally, the proximal edge 138e will dig into the catheter 200 and move distally with the catheter 200 and more firmly retain the catheter 200 on the tunneling device 100.

Referring again to FIG. 1, tunneling cap 140, which may operate as a tissue dissector or expander, includes a substantially bullet-shaped body 142 that facilitates passage of tunneler device 100 through subcutaneous tissue during a tunneling procedure. Body 142 includes internal threads 144 at a proximal end and a substantially rounded atraumatic tip 146 at a distal end. As discussed above, internal threads 144 of body 142 mate with external threads 134a of threaded segment 134 of connecting assembly 130 to releasably secure tunneling cap 140 to connecting assembly 130. With tunneling cap 140 secured to connecting assembly 130, extension segment 136 and lariat member 138 are enclosed within tunneling cap 140 to facilitate passage of the tunneler device 100 through the subcutaneous tissue. Although tunneling cap 140 and connecting assembly 130 are configured for threaded engagement with each other, any suitable arrangement may be utilized to releasably secure tunneling cap 140 to connecting assembly 130. For example, tunneling cap 140 may be secured to connecting assembly 130 with a bayonet coupling or via friction fit arrangement. Tunneling cap 140 may be provided in various sizes for creating tunnels of different sizes. In this manner, tunneler device 100 may be used for the implantation of catheters having different sizes. Tunneling cap 140 may include longitudinal ribs 148 which extend along at least a portion of a length of body 142. Ribs 148 may be configured to facilitate manipulation of tunneling cap 140 by a clinician during securement and removal of tunneling cap 140 relative to connecting assembly 130.

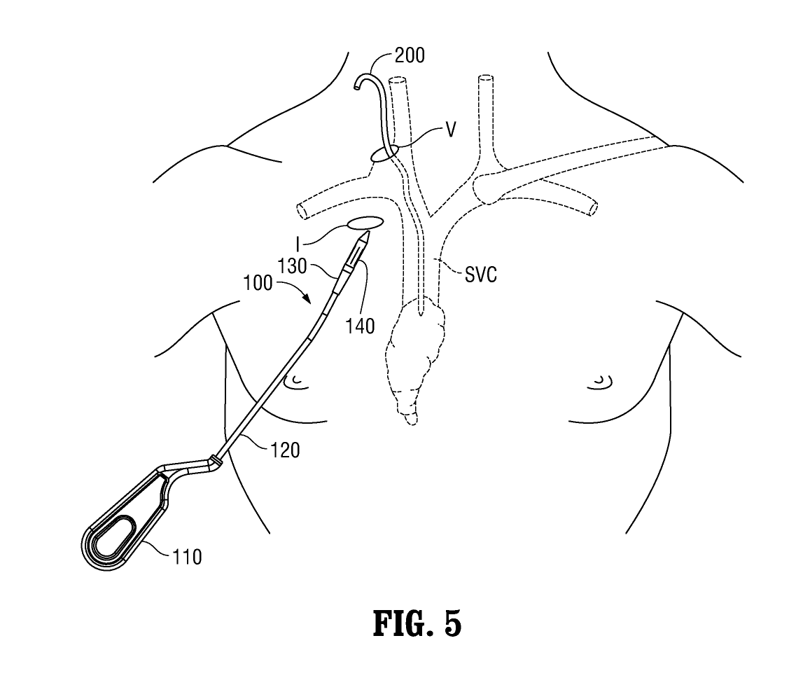

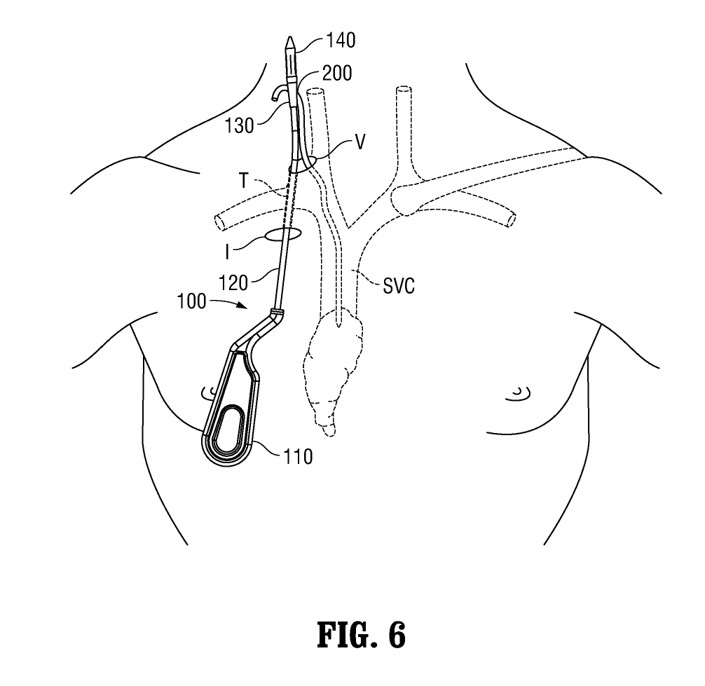

Referring to FIGS. 5-8, the use of tunneler device 100 will now be described with respect to an exemplary procedure. In use, after catheter 200 has been positioned into a patient's vasculature, for example, the superior vena cava “SVC”, such that a proximal end of catheter 200 extends from the venotomy site “V” (FIG. 5), a subcutaneous tunnel “T” (FIG. 6) is formed between the venotomy site “V” and a position adjacent a clavicle of the patient. To create the tunnel “T”, an incision “I” is made adjacent the clavicle and the distal end of tunneler device 100, with cap 140 attached, is inserted through incision “I” and pushed through tissue to venotomy site “V.”

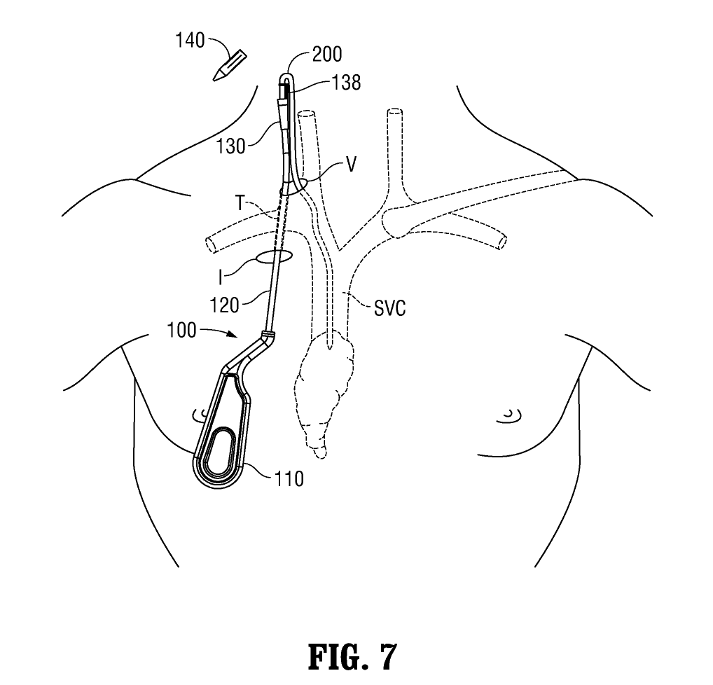

After the distal end of tunneler 100 is positioned to extend from venotomy site “V”, cap 140 is removed from the distal end of tunneler device 100 to expose connecting assembly 130 and extension segment 136 of connecting assembly 130 is inserted into the lumen 202 at the proximal end of catheter 200 (FIG. 7). With reference to FIG. 4A, tapering distal surface 138c of capturing element 138b enables catheter 200 to be slid into opening 137 in the direction of arrow “A” to position extension segment 136 of connecting assembly 130 within a lumen of catheter 200. When connecting assembly 130 and catheter 200 are secured together, proximal edge 138e of capturing element 138b obstructs catheter 200 from being slid away from lariat member 138 in the direction of arrow “B.” In addition, the plurality of barbs 136a of connecting assembly 130 are frictionally engaged with the inner surface of catheter 200 to further secure the tunneling device 100 to the catheter 200.

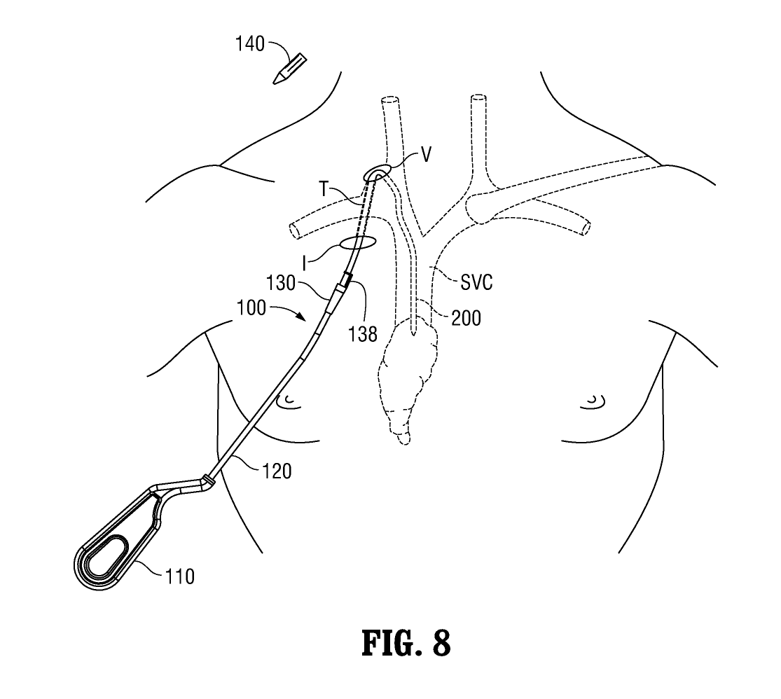

As best shown in FIG. 8, once catheter 200 is secured to tunneler device 100 through the engagement of connecting assembly 130 with the proximal end of catheter 200, tunneler device 100 is drawn through tunnel “T” such that the proximal end of catheter 200 extends outwardly from incision “I.” Catheter 200 may then be disconnected from tunneling device 100. In particular, the proximal end of catheter 200 may be cut to disengage the tunneler device 100 from the rest of catheter 200. As discussed above, any or all of tunneler device 100 may be disposable or sterilizable and reusable.

As can be appreciated, the tunneler device of the present disclosure and any of its components may be formed of any suitable polymeric or metallic material and combinations thereof. Further, while a reverse tunneling procedure has been described, the disclosed tunneler device 100 may be used in any tunneling procedure, such as a retrograde tunneling procedure by replacing the handle 110 with a trocar.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Accordingly, the subject matter of the present disclosure is not to be limited by what has been particularly shown and described.

1. A tunneler device, comprising:
a handle;
a shaft extending distally from the handle; and
a connecting assembly including a body positioned on a distal end of the shaft, the body supporting an extension segment and a lariat member, the extension segment being configured to be received in a lumen of a catheter for engagement with an inner surface of the catheter, the lariat member including a rod and a capturing element, the rod extending distally from the body and supporting the capturing element, the capturing element being configured to be at least partially positioned about a circumference of an outer surface of the catheter and being configured to enable movement of the catheter to a position about the extension segment but to obstruct movement of the catheter from about the extension segment.

2. The tunneler device according to claim 1, wherein the rod of the lariat member and the extension segment define parallel axes.

3. The tunneler device of claim 2, wherein the capturing element of the lariat member encircles at least a portion of the extension segment and is spaced from the extension segment.

4. The tunneler device of claim 1, wherein the capturing element includes an inwardly tapering distal surface that defines an opening configured and dimensioned to enable movement of the catheter about the extension segment.

5. The tunneler device of claim 4, wherein the capturing element defines a proximal edge positioned to engage the catheter to obstruct movement of the catheter from the extension segment.

6. The tunneler device of claim 4, wherein the opening is substantially circular.

7. The tunneler device of claim 1, further comprising a cap that is selectively engageable with the connecting assembly.

8. The tunneler device of claim 7, wherein the body of the connecting assembly includes a threaded segment with an external thread and the cap includes an internal thread such that the internal thread of the cap and the external thread of the threaded segment of the body are selectively threadably engageable to secure the cap to the connecting assembly.

9. The tunneler device of claim 8, wherein the cap encloses the extension segment and the lariat member when the cap is secured to the connecting assembly.

10. A tunneler device, comprising:
a handle;
a shaft supported on distal end of the handle; and
a body positioned on a distal end of the shaft, the body supporting an extension segment and a lariat member, the lariat member including a capturing element supported by the body, the capturing element being positioned at least partially about a perimeter of the extension segment in spaced apart relationship with the extension segment, wherein the extension segment and the capturing element define an at least partial annular channel dimensioned to receive a catheter.

11. The tunneler device according to claim 10, wherein the capturing element is supported by the body by a rod extending from the body, and the rod and the extension segment define parallel axes.

12. The tunneler device of claim 10, wherein the capturing element includes a distal inwardly tapering surface that defines an opening configured and dimensioned to receive the catheter.

13. The tunneler device of claim 12, wherein the opening is substantially circular.

14. The tunneler device of claim 10, wherein the capturing element defines a proximal edge positioned to engage the catheter to obstruct movement of the catheter from the extension segment.

15. The tunneler device of claim 10, further comprising a cap that is selectively engageable with the body.

16. The tunneler device of claim 15, wherein the body includes a threaded segment with an external thread and the cap includes an internal thread such that the internal thread of the cap and the external thread of the threaded segment of the body are selectively threadably engageable to secure the cap to the body.

17. The tunneler device of claim 15, wherein the cap encloses the extension segment and the lariat member when the cap is secured to the body.

18. A tunneler device, comprising:
a handle;
a shaft extending distally from the handle; and
a connecting assembly including a body positioned on a distal end of the shaft, the body supporting an extension segment and a lariat member, the extension segment being configured to be received in a lumen of a catheter for engagement with an inner surface of the catheter, the lariat member including a rod and a capturing element, the rod extending distally from the body and supporting the capturing element, the capturing element configured to surround an outer surface of the catheter and includes a distal inwardly tapering surface and a proximal edge positioned configured to engage the catheter to obstruct movement of the catheter from the extension segment.

19. The tunneler device of claim 18, wherein the tunneler device includes a cap that is selectively attachable to the body of the connecting assembly to enclose the extension segment and the lariat member for facilitating passage of the tunneler device through a subcutaneous tunnel.

20. The tunneler device of claim 19, wherein the cap encloses the extension segment and the lariat member when the cap is secured to the body.
